(19) United States
(12) Patent Application Publication
(10) Pub. No.: US 2023/0056618 A1
(43) Pub. Date: Feb. 23, 2023

(54) CMV RESISTANCE CONFERRING GENES

(71) Applicant: RIJK ZWAAN ZAADTEELT EN ZAADHANDEL B.V., De Lier (NL)

(72) Inventors: Cornelis VAN DER MAAS, De Lier (NL); Adrianus Cornelis KOEKEN, De Lier (NL)

(21) Appl. No.: 17/875,549

(22) Filed: Jul. 28, 2022

Related U.S. Application Data
(63) Continuation-in-part of application No. PCT/EP2021/054929, filed on Feb. 26, 2021.
Application No. PCT/EP2021/054929 is a continuation-in-part of application No. PCT/EP2020/055065, filed on Feb. 26, 2020.

(51) Int. Cl. C12N 15/82; C12Q 1/6895; C07K 14/415

(52) U.S. Cl. CPC C12N 15/8283; C12Q 1/6895; C07K 14/415; C12Q 2600/13; C12Q 2600/156

(57) ABSTRACT

The present invention relates to a modified ABCB9 gene encoding a protein conferring resistance to CMV in a plant of the Cucurbitaceae, such as a cucumber plant, in which the protein is expressed, where the gene has a) a nucleotide sequence which encodes a protein with SEQ ID NO: 4; b) a nucleotide sequence with SEQ ID NO: 3; c) a nucleotide sequence encoding a protein derived by substitution, deletion and/or addition of one or more amino acids of the protein with SEQ ID NO: 4; d) a nucleotide sequence that encodes a protein with an amino acid sequence, which is at least 85% identical to SEQ ID NO: 4; e) a nucleotide sequence which is at least 85% identical to SEQ ID NO: 3; or f) a nucleotide sequence according to c) or d) wherein the protein has a methionine (M) on position 428 of SEQ ID NO: 4, or on a position corresponding thereto.

### RELATED APPLICATIONS AND INCORPORATION BY REFERENCE

This application is a continuation-in-part application of international patent application Serial No. PCT/EP2021/054929 filed 26 Feb. 2021, which published as PCT Publication No. WO 2021/170850 on 2 Sep. 2021, which claims benefit of international patent application Serial No. PCT/EP2020/055065 filed 26 Feb. 2020.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The instant application contains a Sequence Listing which has been submitted electronically and is hereby incorporated by reference in its entirety. Said ASCII copy was created Jul. 26, 2022, is named ReplacementY7954-00525SL.xml and is 30,744 bytes in size.

### FIELD OF THE INVENTION

The present invention relates to a modified plant gene in Cucurbitaceae that increases the resistance of a plant to CMV, a plant having the modified gene, and to methods for identifying, selecting and developing such a plant. The invention further relates to markers for identifying CMV resistant plants.

### BACKGROUND OF THE INVENTION

The Cucumber Mosaic virus (CMV) is a widely spread disease and it affects many plant species from up to 100 different plant families among which the Cucurbits or Cucurbitacea. It belongs to the genus Cucumovirus and to the virus family of Bromoviridae. CMV is mainly transmitted by aphids although it can be spread mechanically by humans too. The virus was firstly identified in cucumber in 1934. Plant tissue that is infected shows characteristic viral inclusion bodies which can be used to diagnose the pathogen. The inclusion bodies are hexagonal in shape, or rhomboidal, may appear hollow and can aggregate together to form larger spots. Other symptoms may be leaf mosaic or mottling, yellowing, ringspots, stunting and distortion of flowers, leaf and fruit. In cucumbers, an infection of CMV can cause cucumber leaves to turn mosaic, wrinkled and misshapen, the growth of the plants is stunted and the cucumber fruits are often oddly shaped, greyish and taste bitter.

Although some sources of CMV resistance in Cucurbitacea like cucumber and melon are known to exist, so far no gene(s) have been described to be responsible for the genetic basis of CMV resistance.

Citation or identification of any document in this application is not an admission that such document is available as prior art to the present invention.

### SUMMARY OF THE INVENTION

It is an object of the present invention to provide one or more genes that are related to CMV resistance.

In the research leading to the current invention, it was found that the presence of a modification in the ABCB9 gene confers CMV resistance in a plant compared to a plant which may comprise the wild type of the ABCB9 gene. The ABCB9 gene encodes a protein called ABC transporter B family member 9, and is part of the ABC transporter superfamily that encodes proteins that are supposedly involved in ATPase-coupled transmembrane transporter activity.

The present invention provides a modified ABCB9 gene encoding a protein conferring resistance to CMV in a plant of the Cucurbitaceae, in particular a cucumber plant, in which the protein is expressed, characterized in that the gene may comprise a nucleotide sequence selected from

a) a nucleotide sequence which encodes a protein which may comprise SEQ ID NO: 4;

b) a nucleotide sequence which may comprise SEQ ID NO: 3;

c) a nucleotide sequence encoding a protein derived by substitution, deletion and/or addition of one or more amino acids of the protein which may comprise SEQ ID NO: 4;

d) a nucleotide sequence that encodes a protein which may comprise an amino acid sequence, which is at least 85% identical to SEQ ID NO: 4;

e) a nucleotide sequence which is at least 85% identical to SEQ ID NO: 3;

f) a nucleotide sequence according to c) or d) wherein the protein may comprise a methionine (M) on position 428 of SEQ ID NO: 4 or a on position corresponding thereto.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of’ and “consists essentially of have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1: The nucleotide sequences of the ABCB9 gene, protein sequences encoded by the ABCB9 gene, wild type and modified versions. (SEQ ID NO: 1 - SEQ ID NO: 4).

FIG. 2: The nucleotide sequences of the EF1-a gene, protein sequences encoded by the EF1-α gene, wild type and modified versions. (SEQ ID NO: 5 - SEQ ID NO: 8).

### DETAILED DESCRIPTION OF THE INVENTION

The present invention provides a modified ABCB9 gene encoding a protein conferring resistance to CMV in a plant of the Cucurbitaceae, in particular a cucumber plant, in which the protein is expressed, characterized in that the gene may comprise a nucleotide sequence selected from

a) a nucleotide sequence which encodes a protein which may comprise SEQ ID NO: 4;

b) a nucleotide sequence which may comprise SEQ ID NO: 3;

c) a nucleotide sequence encoding a protein derived by substitution, deletion and/or addition of one or more amino acids of the protein which may comprise SEQ ID NO: 4;

d) a nucleotide sequence that encodes a protein which may comprise an amino acid sequence, which is at least 85% identical to SEQ ID NO: 4;

e) a nucleotide sequence which is at least 85% identical to SEQ ID NO: 3;

f) a nucleotide sequence according to c) or d) wherein the protein may comprise a methionine (M) on position 428 of SEQ ID NO: 4 or a on position corresponding thereto.

A SNP was identified in the ABCB9 gene of CMV resistant cucumber plants. The modification in the ABCB9 gene of the invention may comprise a substitution of the nucleotide on position 1282 in the wild type ABCB9 gene nucleotide sequence of SEQ ID NO: 1.

The SNP was identified at position 1282 in wild type ABCB9 gene nucleotide sequence SEQ ID NO: 1 and constitutes a guanine (G) in the CMV susceptible wild type of the ABCB9 gene and an adenine (A) in the CMV resistant modified ABCB9 gene.

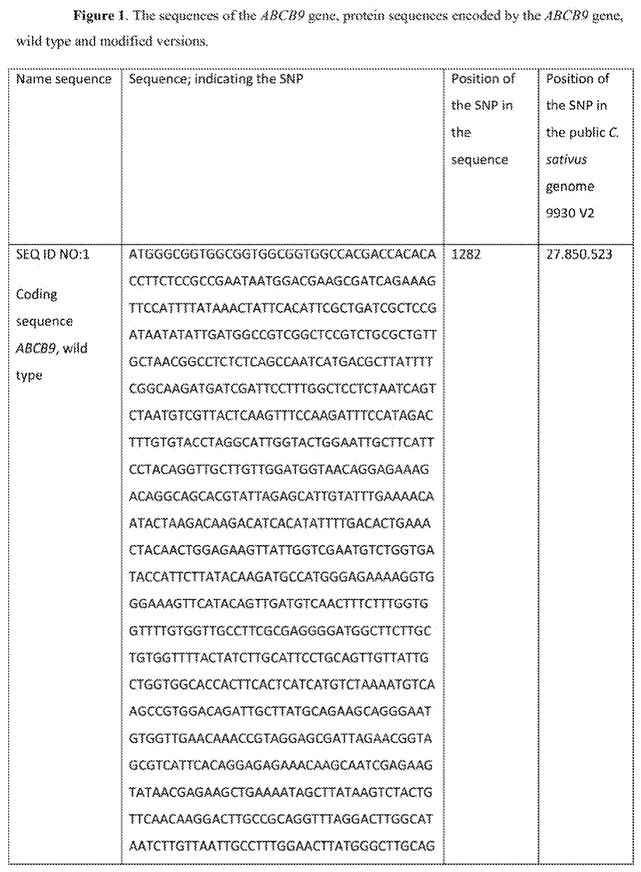

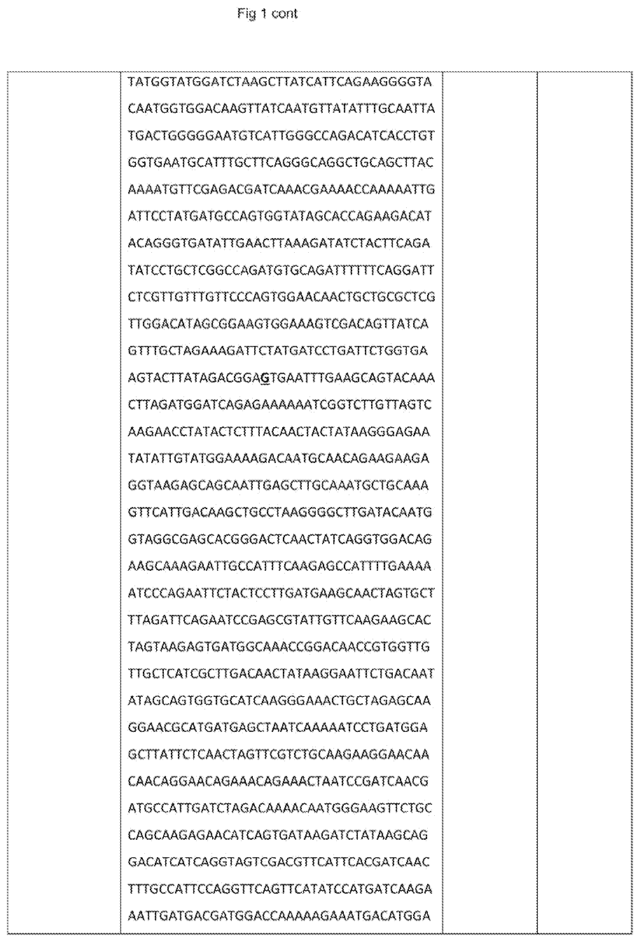

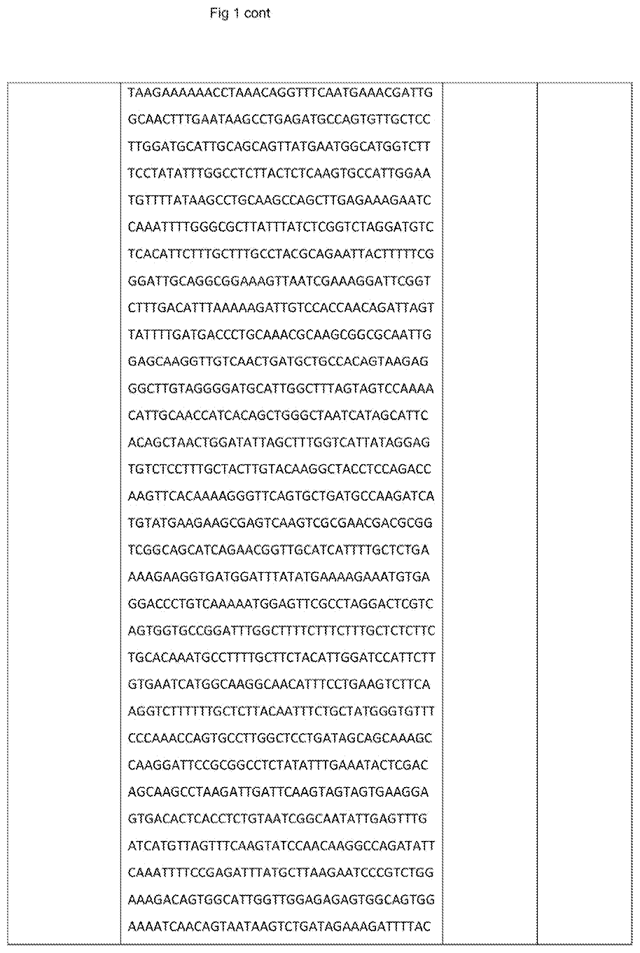

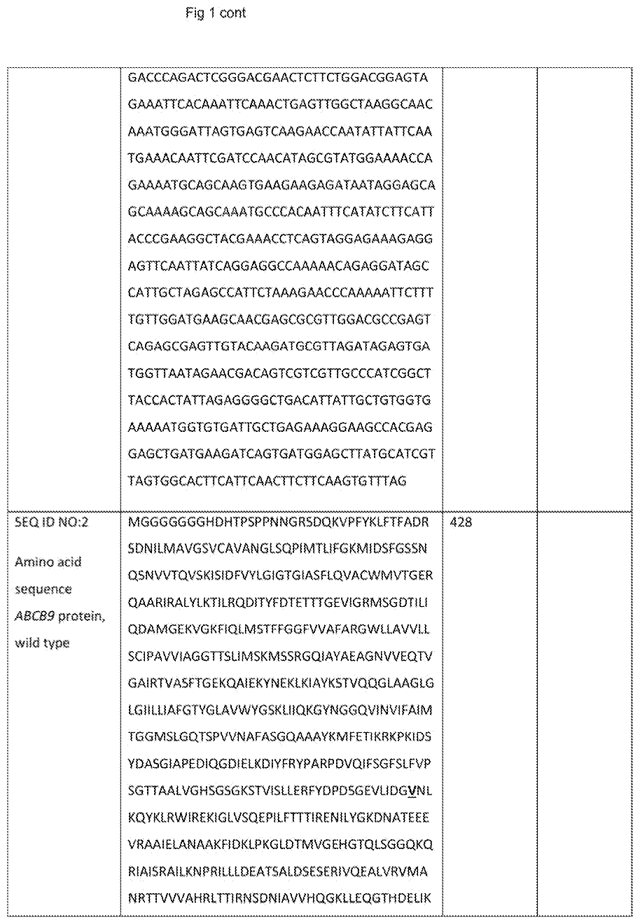

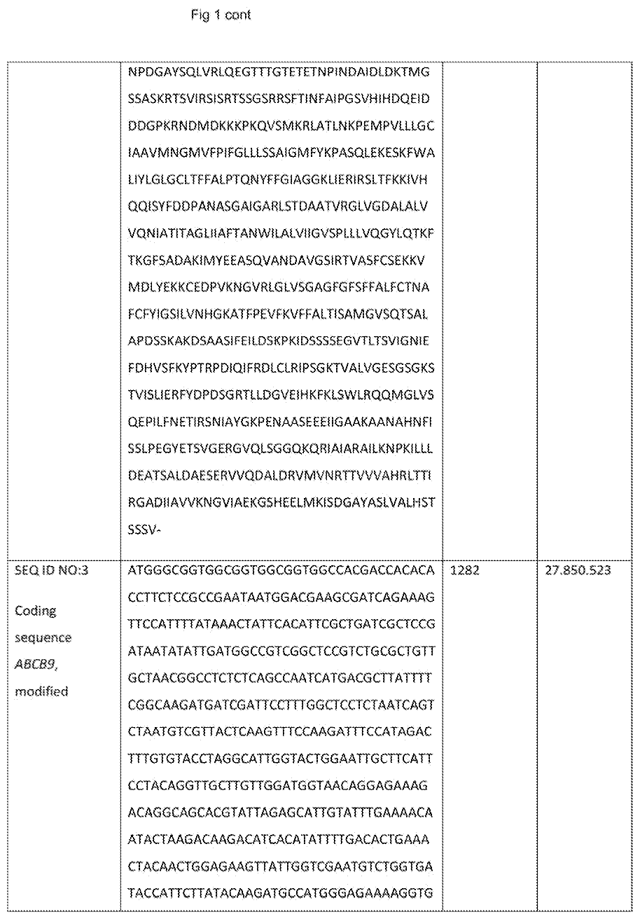

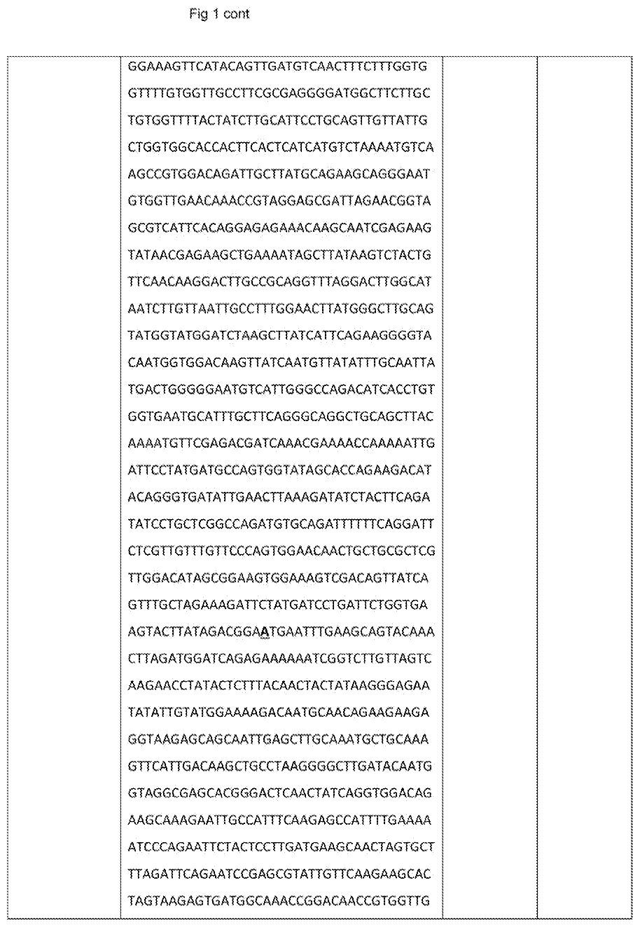

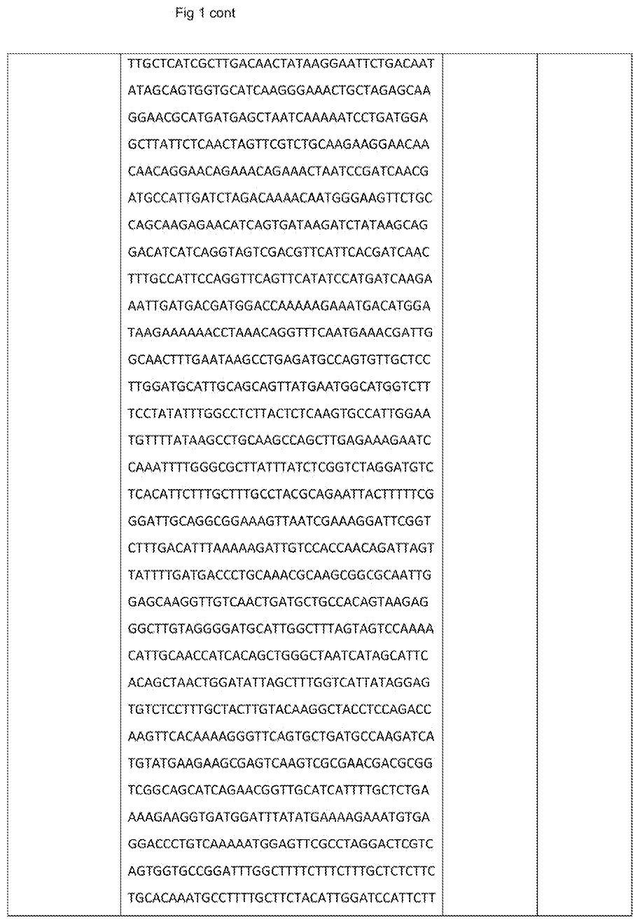

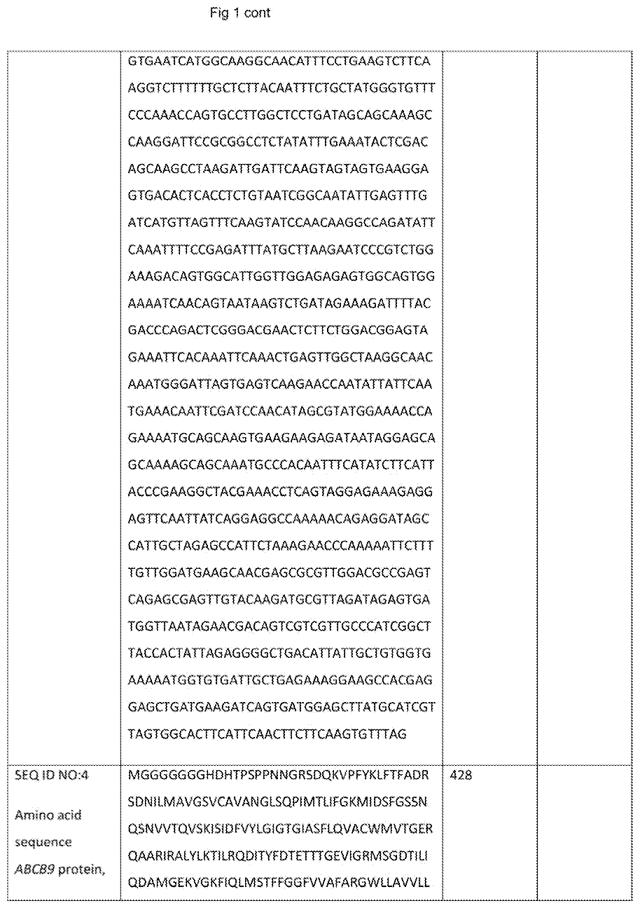

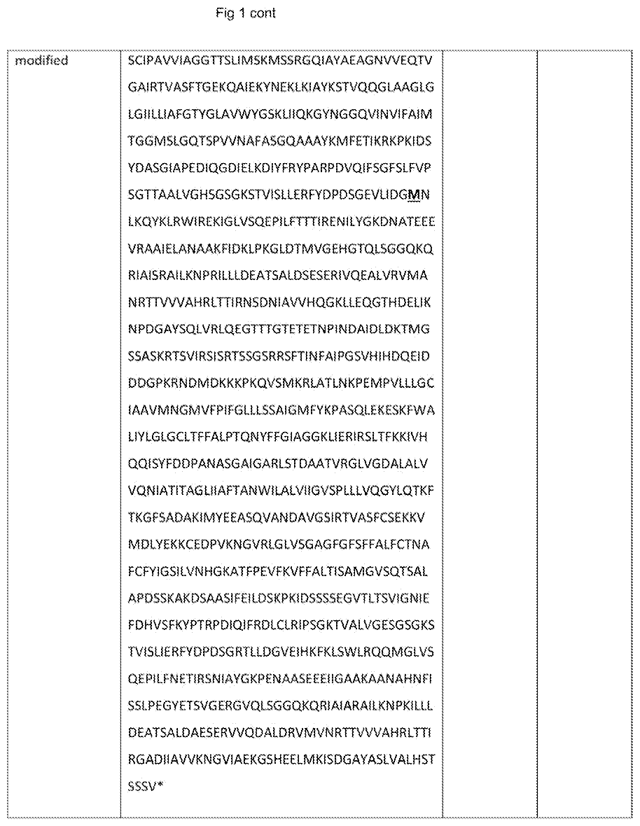

The modification in the wild type ABCB9 gene nucleotide sequence SEQ ID NO: 1 leads to a change in the wild type amino acid sequence of SEQ ID NO: 2, shown in FIG. 1.

The modification on position 1282 of nucleotide sequence SEQ ID NO: 1 leads to a substitution of amino acid Valine (V), on position 428 of the wild type amino acid sequence SEQ ID NO: 2, to amino acid Methionine (M), as presented in the modified amino acid sequence SEQ ID NO: 4, shown in FIG. 1. The position of the SNP lies in the socalled Md1B domain. The Md1B is a conserved protein domain family that is involved in the activities of the ABC-type multidrug transport system, ATPase and permease component.

The modified ABCB9 gene of the invention confers CMV resistance when homozygously present in a plant of the Cucurbitaceae, in particular a cucumber plant.

As used herein, the ABCB9 gene is a gene encoding the ABCB9 protein. As used herein, the ABCB9 gene is a gene which may comprise a wildtype coding sequence represented by SEQ ID NO: 1, or a homologous gene which may comprise a nucleotide sequence having at least 85% sequence identity to SEQ ID NO: 1, or a gene encoding a ABCB9 protein which may comprise SEQ ID NO: 2, or a gene encoding a homologous ABCB9 protein which may comprise an amino acid sequence having at least 85% sequence identity to SEQ ID NO: 2.

A homologous ABCB9 gene may comprise a sequence having at least 85% sequence identity to SEQ ID NO: 1, preferably 86%, 87%, 88%, 89%, 90%, 91%, 92%, 93%, 94%, 95%, 96%, 97%, 98%, or 99%. A homologous ABCB9 protein may comprise an amino acid sequence having at least 85% sequence identity to SEQ ID NO: 2, preferably 86%, 87%, 88%, 89%, 90%, 91%, 92%, 93%, 94%, 95%, 96%, 97%, 98%, or 99%.

As used herein, sequence identity is the percentage of nucleotides or amino acids that is identical between two sequences after aligning those sequences. The person skilled in the art is aware of how to align sequences, for example by using a sequence alignment tool such as BLAST.

The modified ABCB9 gene as used herein, refers to a modified nucleotide sequence of the ABCB9 gene as compared to the wild type nucleotide sequence of the ABCB9 gene. The change or modification can be any change or modification, including but not limited to a nucleotide substitution.

The invention also provides a protein, conferring resistance to CMV in a plant of the Cucurbitaceae, in particular a cucumber plant, in which the protein is expressed and which protein is encoded by the modified ABCB9 gene as described.

In the research leading up to the invention, it was found that apart from a modified ABCB9 gene, also a modified EF1-α gene confers CMV resistance when homozygously present in the genome of a cucumber plant. The EF1-α gene encodes a protein called Elongation transcription factor 1-alpha, and is part of the GTP-binding elongation factor family of genes that are supposedly involved in protein synthesis and developmental control. A SNP was identified at position 1115 in the wild type EF1-α gene nucleotide sequence SEQ ID NO: 5 and constitutes a cytosine (C) in the CMV susceptible wild type of the EF1-α gene and a guanine (G) in the CMV resistant modified EF1-α gene. The position of the SNP of the invention lies in the translation elongation factor 1 (TEF-1) protein domain family. Elongation factor 1 plays a role in the protein synthesis and interacts as host factor with potential pathogens.

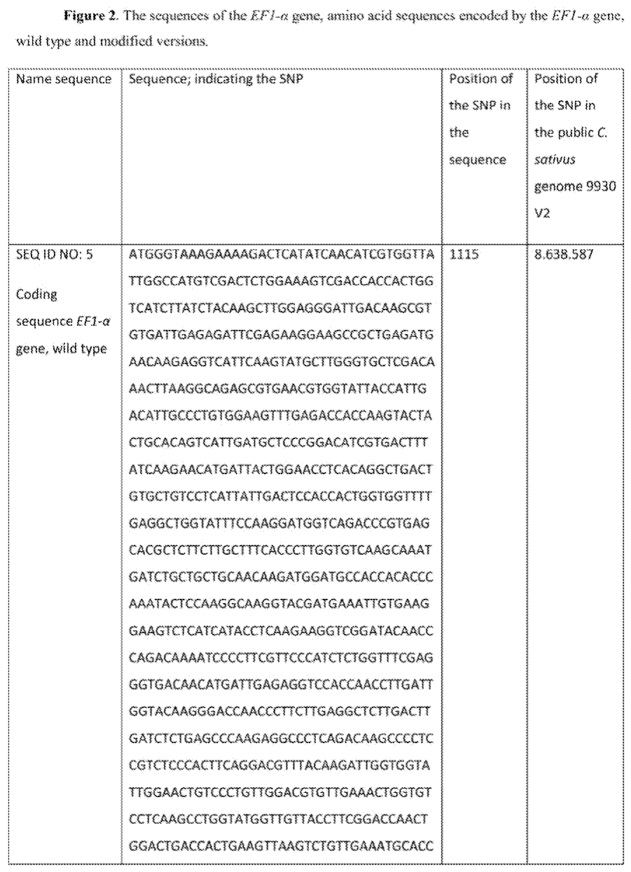

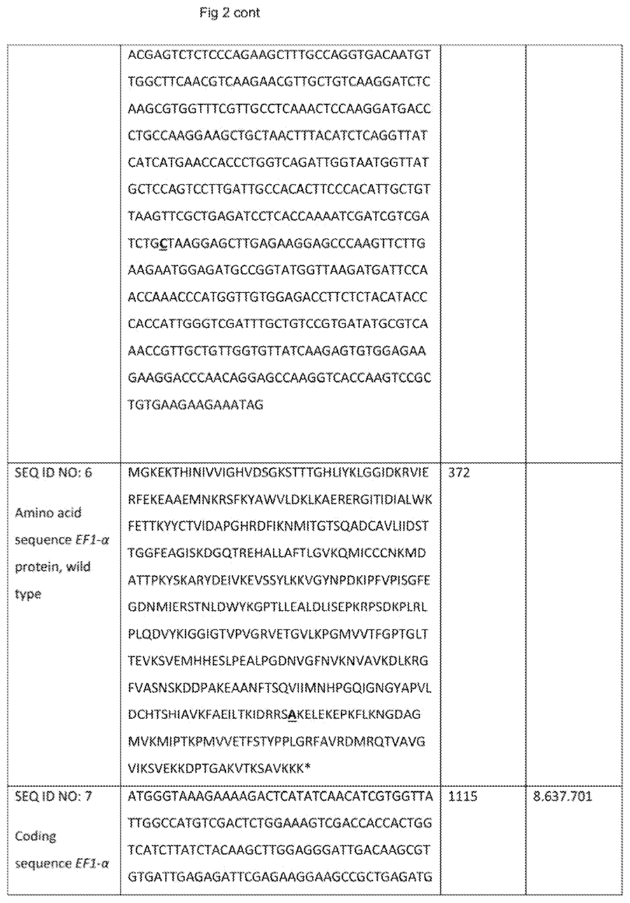

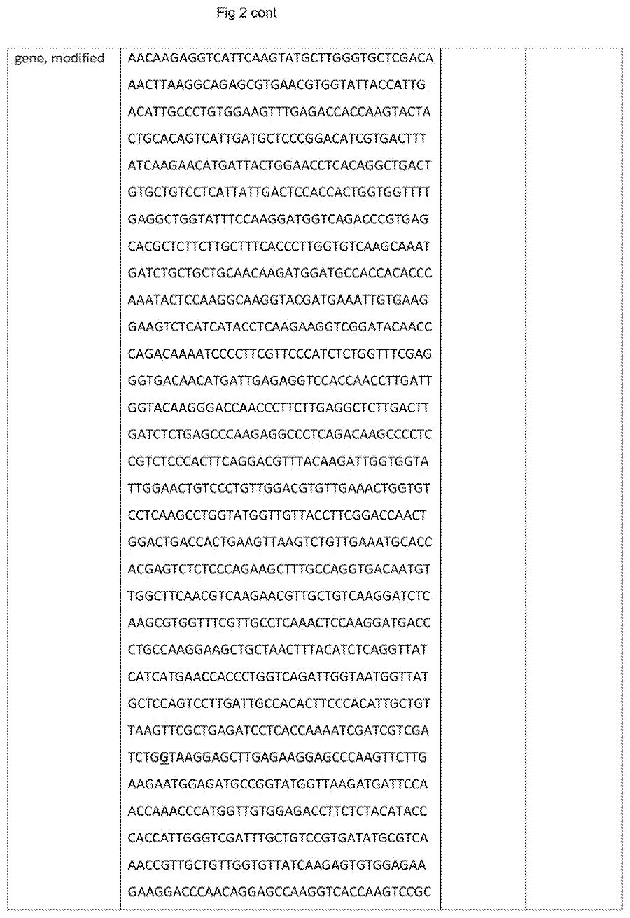

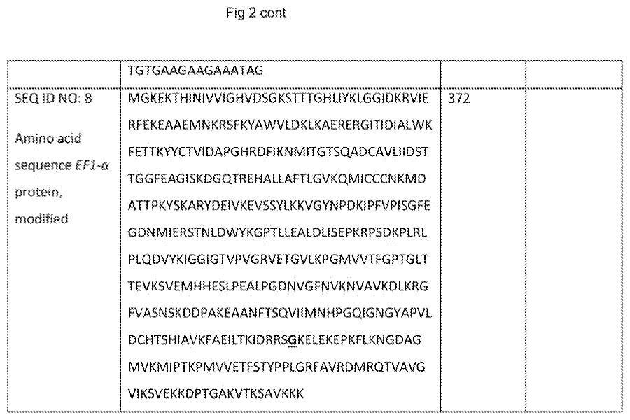

The modified EF1-α gene may comprise SEQ ID NO: 7. The modification in the wild type EF1-α gene nucleotide sequence SEQ ID NO: 5 leads to a change in the wild type amino acid sequence of SEQ ID NO: 6. The modification in the wild type amino acid sequence of SEQ ID NO: 6 leads to modified amino acid sequence SEQ ID NO: 8, both shown in FIG. 2.

The modification on position 1115 of nucleotide sequence SEQ ID NO: 5 leads to a substitution of amino acid alanine (A), on position 372 of the wild type amino acid sequence of SEQ ID NO: 6 to amino acid glycine (G), in the modified amino acid sequence as presented by SEQ ID NO: 8.

The present invention provides a modified EF1-α gene encoding a protein conferring resistance to CMV in a plant of the Cucurbitaceae, in particular a cucumber plant, in which the protein is expressed, characterized in that the gene may comprise a nucleotide sequence selected from

a) a nucleotide sequence which encodes a protein which may comprise SEQ ID NO: 8,

b) a nucleotide sequence which may comprise SEQ ID NO: 7;

c) a nucleotide sequence encoding a protein derived by substitution, deletion and/or addition of one or more amino acids of the protein which may comprise SEQ ID NO: 8,

d) a nucleotide sequence that encodes a protein which may comprise an amino acid sequence, which is at least 95% identical to SEQ ID NO: 8, or

e) a nucleotide sequence which is at least 95% identical to SEQ ID NO: 7;

f) a nucleotide sequence according to c) or d) wherein the protein may comprise a glycine (G) on position 372 of SEQ ID NO: 8 or on a position corresponding thereto.

The modified EF1-α gene as described, confers CMV resistance when homozygously present in a plant of the Cucurbitaceae, in particular a cucumber plant. The modified EF1-α gene as used herein refers to a modified nucleotide sequence of the EF1-α gene as compared to the wild type nucleotide sequence of the EF1-α gene. The change or modification can be any change or modification, including but not limited to a nucleotide substitution. The wild type nucleotide sequence of the EF1-α gene is shown by SEQ ID NO: 5, the modified nucleotide sequence of the EF1-α gene is shown in SEQ ID NO: 7, see FIG. 2.

As used herein, the EF1-α gene is a gene encoding the EF1-α protein. As used herein, the EF1-α gene is a gene which may comprise a wildtype coding sequence represented by SEQ ID NO: 5, or a homologous gene which may comprise a sequence having at least 95% sequence identity to SEQ ID NO: 5, or a gene encoding a EF1-α protein which may comprise SEQ ID NO: 6, or a gene encoding a homologous EF1-α protein which may comprise a sequence having at least 95% sequence identity to SEQ ID NO: 6.

A homologous EF1-α gene may comprise a sequence having at least 95% sequence identity to SEQ ID NO: 5, preferably 96%, 97%, 98%, or 99%. A homologous EF1-α protein may comprise a sequence having at least 95% sequence identity to SEQ ID NO: 6, preferably 96%, 97%, 98%, or 99%.

The invention also relates to a protein, conferring resistance to CMV in a plant of the Cucurbitaceae, in particular a cucumber plant, in which the protein is expressed and which protein is encoded by the modified EF1-α gene as described.

The EF1-α protein of the invention does not comprise one or more of the following: an isoleucine at position 259, a valine at position 293, a serine at position 405. The nucleotide sequence encoding the protein does not have a codon encoding an isoleucine at position 259, a valine at position 293, a serine at position 405 of the protein. The invention further relates to a plant of the Cucurbitaceae, in particular a cucumber plant, or a part thereof, which may comprise the modified ABCB9 gene as described in the present application.

A plant of the invention is a plant of the family Cucurbitaceae, in particular a cucumber plant, Cucumis sativus, most preferably an agronomically elite Cucumis sativus plant.

In the context of this invention, an agronomically elite plant is a plant having a genotype that, as a result of directed crossing and selection by human interventions, may comprise an accumulation of distinguishable and desirable agronomic traits which allow a producer to harvest a product of commercial significance.

The plant of the invention may be a plant of an inbred line, a hybrid, a doubled haploid, or a plant of a segregating population. As used herein, a plant of an inbred line is a plant of a population of plants that is the results of three or more rounds of selfing, or backcrossing; or which plant is double haploid. An inbred line may for example be a parent line used for production of a commercial hybrid.

The plant of the invention, a plant of the family of Cucurbitaceae, in particular a cucumber plant, preferably may comprise the modified ABCB9 gene homozygously. When the plant may comprise the modified ABCB9 gene homozygously, it shows resistance to CMV. When the plant of the invention may comprise the modified ABCB9 gene heterozygously, it may be crossed or selfed to produce a plant that may comprise the modified ABCB9 gene homozygously and shows resistance to CMV.

The invention also relates to a plant of the Cucurbitaceae, in particular a cucumber plant, or a part thereof, which may comprise the modified EF1-α gene as described herein.

The invention also provides a plant of the Cucurbitaceae, in particular a cucumber plant, or a part thereof, which may comprise both the modified ABCB9 gene and the modified EF1-α gene. Each of the modified genes of the invention on its own, provided that it is homozygously present in the genome, gives resistance to CMV. However, a plant which may comprise both modified genes of the invention homozygously, shows a higher resistance to CMV, as compared to a plant which may comprise only one of said modified genes homozygously. This is illustrated in Example 1.

As used herein, resistance to CMV is defined as resistance to the Cucumber Mosaic Virus, also abbreviated as CMV. As used herein, the presence of CMV resistance can be determined by performing a bio-assay.

For the bio-assay, cucumber seeds are sown and after germination the seedlings are grown for 6 days in a greenhouse. On day 7 after sowing, at least 20 individual plants for each line are inoculated. On day 9 after sowing, the inoculation is repeated. Just before the inoculation, the plants are dusted with carborundum powder. Preparing the inoculum, fresh CMV infected leaves are ground and suspended in a phosphate buffer using a ratio of 3 ml of buffer for each gram of plant material. During the preparation of the inoculum, the buffer and the inoculum are being cooled.

The inoculation itself is done by rubbing the leaves with a sponge that was dipped in the suspension made with the infected plant material. Directly after the inoculation, the plants are rinsed with plenty of water. The test CMV isolate that is used is known as CU-CMV-UK6 and is available from PRI-WUR in Wageningen.

The plants are assessed for symptoms of CMV infection according to classes 1 to 6, as presented in Table 2, first on day 21 after sowing, and a second time on day 28 after sowing., The second leaf of each individual plants is assessed for symptoms. For each group/line, all the individual plant scores are collected and averaged.

Further details of the bio-assay can be found in Example 1. As a CMV susceptible control in the described bio-assay, plants of the variety Ventura RZ are used. In Table 2 is described for each of the scores or classes for CMV resistance which phenotypic symptoms a plant leaf should show to receive such a score. The CMV resistance score as given in Table 2 has a range of 1 to 6, with a low score representing resistance to CMV and a high score representing susceptibility to CMV. A cucumber plant with a CMV resistance score of lower than 3.0 is defined as resistant to CMV, a CMV resistance score of 3 or higher is defined as susceptible.

A cucumber plant comprising the wildtype ABCB9 gene and the wildtype EF1-α gene homozygously show an average CMV resistance score of 3.0 or higher. A cucumber plant comprising the modified EF1-α gene homozygously shows an average CMV resistance score lower than 3.0, preferably lower than 2.5, and a cucumber plant comprising the modified ABCB9 gene homozygously shows an average CMV resistance score lower than 2.5, preferably lower than 2.0. If modified genes ABCB9 and EF1-α are both homozygously present, the average CMV resistance score is lower than 2.0, preferably lower than 1.5. The results are shown in Table 3.

The use of two or more resistance genes in one crop to manage plant diseases, also known as ‘gene stacking’, is generally more durable than using only one resistance gene or using two single-gene resistance varieties, as different studies have shown.

The plant of the invention preferably is a plant which may comprise the modified ABCB9 gene homozygously and optionally which may comprise the modified EF1-α gene homozygously, wherein the cucumber plant is resistant to CMV.

The invention also relates to a part of a plant of the Cucurbitaceae, preferably a part of a plant of cucumber, which plant part may comprise the modified ABCB9 gene of the invention, and optionally the modified EF1-α gene of the invention. Preferably the plant part may comprise the modified ABCB9 gene homozygously and optionally may comprise the modified EF1-α gene homozygously, wherein the cucumber plant that can be grown from the plant part is resistant to CMV.

The invention also relates to a cucumber seed which may comprise the modified ABCB9 gene of the invention, and/or the modified EF1-α gene of the invention, preferably a seed which may comprise the modified gene(s) homozygously. The seed as described herein is also referred as “the seed of the invention”. A cucumber plant grown from this seed may comprise the modified ABCB9 gene and/or the modified EF1-α gene of the invention and is thus a plant of the invention.

The invention also covers seed produced by a plant of the invention. These seed comprise a modified ABCB9 gene and/or modified EF1-α gene and therefore a plant grown from said seed is a plant of the invention. If the plant of the invention grown from the seed of the invention may comprise the modified ABCB9 gene homozygously and/or modified EF1-α gene homozygously, the plant will show resistance to CMV.

The invention further relates to propagation material derived from a plant, plant material or seed of the invention, and which may comprise the modified ABCB9 gene and/or the modified EF1-α gene of the invention. The propagation material is selected from a microspore, pollen, an ovary, an ovule, an embryo, an embryo sac, an egg cell, a cutting, a root, a hypocotyl, a cotyledon, a stem, a leaf, a flower, an anther, a seed, a meristematic cell, a protoplast, or a cell, or a tissue culture thereof. The propagation material can comprise the modified ABCB9 gene and/or the modified EF1-α gene either heterozygously or homozygously.

The invention also relates to a cell of a plant of the invention. Said cell may be a cell in isolated condition or as a part of a complete plant or plant parts thereof. A cell of the plant of the invention may comprise the genetic information, which in the current invention is the presence of the modified ABCB9 gene and/or the modified EF1-α gene as defined herein that when homozygously present in the genome of a cell, leads to CMV resistance in a plant of the invention. A cell of the invention may also be a cell that can regenerate into a new plant of the invention.

The invention further relates to plant tissue of a plant of the invention, which may comprise the modified ABCB9 and/or the modified EF1-α gene, of the invention, as described herein. The tissue can be undifferentiated tissue or already differentiated tissue. Undifferentiated tissue can be for example a stem tip, an anther, a petal, or pollen, and can be used in micropropagation to obtain new plantlets that are grown into new plants of the invention. The tissue can also be grown from a cell of the invention.

The use of a cucumber plant as a crop, which cucumber plant may comprise the modified ABCB9 gene and/or the modified EF1-α gene of the invention, preferably homozygously, is considered as part of the invention.

Furthermore, the use of a cucumber plant which may comprise the modified ABCB9 gene and/or the modified EF1-α gene of the invention, preferably homozygously, as a source of seed or as a source of propagation material, is considered as part of the invention.

The invention also relates to the use of a cucumber fruit, which cucumber fruit may comprise the modified ABCB9 gene and/or the modified EFI-α gene of the invention, preferably homozygously, for consumption.

A food product or processed food product, which may comprise the cucumber fruit, or a part thereof, that has the modified ABCB9 gene and/or the modified EFI-α gene of the invention, is also considered part of the invention. The food product may have undergone one or more processing steps. Such a processing step might consist of but is not limited to any of the following treatments or any combination thereof: peeling, cutting, washing, juicing, cooking, cooling. The processed form that may be obtained is also part of the invention.

The invention also provides a marker for identifying a cucumber plant which may comprise a modified ABCB9 gene, wherein the marker detects a modified nucleotide on position 1282 as compared to the wild type ABCB9 gene nucleotide sequence of SEQ TD NO: 1. Preferably the marker of the invention detects an Adenine (A) on position 1282 of SEQ ID NO: 3. Most preferably the marker of the invention may comprise SEQ ID NO: 11, as shown in Table 1.

TABLE 1
The sequences of the markers for
the ABCB9 gene and the EF1-α gene.

| CMV | | |
|---|---|---|
| resistance | | |
| gene | Code | Sequence |
| marker for | SEQ | TGCTGCGCTCGTTGGACATAGCGGAAGTGGAA |
| the wild | ID | AGTCGACAGTTATCAGTTTGCTAGAAAGATTCT |
| type ABCB9 | NO: | ATGATCCTGATTCTGGTGAAGTACTTATAGACG |
| gene | 9 | GAGTGAATTTGAAGCAGTACAAACTTAGATGG |
| | | ATCAGAGAAAAAATCGGTCTTGTTAGTCAAGA |
| | | ACCTATACTCTTTACAACTACTATAAGGGAGAA |
| | | TATATT |
| marker for | SEQ | TGTAGAGAAGGTCTCCACAACCATGGGTTTGGT |
| the wild | ID | TGGAATCATCTTAACCATACCGGCATCTCCATT |
| type | NO: | CTTCAAGAACTTGGGCTCCTTCTCAAGCTCCTT |
| EF1-α gene | 10 | ACCAGATCGACGATCRATTTTGGTGAGGATCTC |
| | | AGCGAACTTAACAGCAATGTGGGAAGTGTGGC |
| | | AATCAAGGACTGGAGCATAACCATTACCAATC |
| | | TGACC |
| marker for | SEQ | TGCTGCGCTCGTTGGACATAGCGGAAGTGGAA |
| the | ID | AGTCGACAGTTATCAGTTTGCTAGAAAGATTCT |
| modified | NO: | ATGATCCTGATTCTGGTGAAGTACTTATAGACG |
| ABCB9 | 11 | GAATGAATTTGAAGCAGTACAAACTTAGATGG |
| gene | | ATCAGAGAAAAAATCGGTCTTGTTAGTCAAGA |
| | | ACCTATACTCTTTACAACTACTATAAGGGAGAA |
| | | TATATT |
| marker for | SEQ | TGTAGAGAAGGTCTCCACAACCATGGGTTTGGT |
| the | ID | TGGAATCATCTTAACCATACCGGCATCTCCATT |
| modified | NO: | CTTCAAGAACTTGGGCTCCTTCTCAAGCTCCTT |
| EF1-α gene | 12 | AGCAGATCGACGATCRATTTTGGTGAGGATCTC |
| | | AGCGAACTTAACAGCAATGTGGGAAGTGTGGC |
| | | AATCAAGGACTGGAGCATAACCATTACCAATC |
| | | TGACC |

The invention also provides a marker for identifying a cucumber plant which may comprise a modified EFI-α gene, wherein the marker detects a modified nucleotide on position 1115 as compared to the wild type EFI-α gene nucleotide sequence of SEQ ID NO: 5. Preferably the marker for identifying a plant which may comprise the modified EE1-α gene detects a Guanine (G) on position 1115 of SEQ ID NO: 7. Most preferably the marker may comprise SEQ ID NO: 12, as shown in Table 1.

The use of a marker for the modified ABCB9 gene and/or the modified EE1-α gene as described herein for identifying a plant that is resistant to CMV is also part of this invention.

The invention further relates to a method for producing a plant that is CMV resistant by introducing the modified ABCB9 gene and optionally introducing the modified EE1-α gene into the genome of a plant. Introducing a (modified) gene can be done by introgression, chemical or physical induced mutagenesis, and so-called gene editing methods.

Modifications or mutations of the ABCB9 gene and/or the modified EE1-α gene can be introduced randomly by means of one or more chemical compounds, such as ethyl methane sulphonate (EMS),nitrosomethylurea, hydroxylamine, proflavine, N-methly-N-nitrosoguanidine, N-ethyl-Nnitrosourea, N-methyl-N-nitro-nitrosoguanidine, diethyl sulphate, ethylene imine, sodium azide, formaline, urethane, phenol and ethylene oxide, and/or by physical means, such as UV-irradiation, fast neutron exposure, X-rays, gamma irradiation, and/or by insertion of genetic elements, such as transposons, T-DNA, retroviral elements.

Modifications of the ABCB9 gene and/or the modified EF1-α gene can also be introduced via more specific, targeted methods like homologous recombination, oligonucleotide-based mutation introduction, zinc-finger nucleases (ZFN), transcription activator-like effector nucleases (TALENs) or Clustered Regularly Interspaced Short Palindromic Repeat (CRISPR) systems.

Modifications can be introduced in seed of a plant of interest in which the CMV resistance is needed. The modification is introduced through mutagenesis, such as an EMS treatment, through radiation, or through a specific targeted approach, such as CRISPR. The skilled person is familiar with these means for introducing modifications into a plant genome. Mutagenized seed is germinated, the resultant plants are selfed or crossed to produce M2 seed.

Subsequently a plant screen is performed to identify the modifications in a ABCB9 and/or a EF1-α gene, based on comparison to the wild type sequence of the ABCB9 gene and/or the EF1-α gene of that plant species.

For Cucumis sativus for example, comparison to SEQ ID NO: 1 for the ABCB9 gene and comparison to SEQ ID NO: 5 for the EF1-α gene can be done. Additionally it should be checked whether the found mutations in the nucleotides lead to changes in the amino acids encoded by the nucleotides. The skilled person is familiar with TILLING to identify mutations in specific genes (McCallum et. al. (2000) Nature Biotechnology, 18: 455-457), and with techniques for identifying nucleotide changes such as DNA sequencing, amongst others. Plants with a modified ABCB9 and/or EF1-α gene are homozygous or made homozygous by selfing, crossing, or the use of doubled haploid techniques which are familiar to the skilled person.

Plants identified and selected on the basis of a modification in a ABCB9 and/or EF1-α gene can then be tested for expressing resistance to CMV. A plant that is produced, identified and selected in this way can thus be confirmed to have its virus resistance as a result from one or more modifications in the ABCB9 and/or EF1-α gene.

Furthermore the invention relates to a method for selecting a CMV resistant plant, may comprise

a) identifying the presence of a modified ABCB9 gene and/or the presence of the modified EF-1 α gene,

b) selecting a plant that may comprise at least one of said modified genes,

c) optionally testing the selected plant for CMV resistance,

d) selecting the plant if it shows CMV resistance as a CMV resistant plant.

The identification of the presence of a modified ABCB9 gene and/or the presence of the modified EF1-α gene is performed by using one or more of the markers of the invention, as defined herein.

Further included in this invention is a method for producing a plant that may comprise the modified ABCB9 gene of the invention, said method which may comprise:

a) crossing a first parent plant which may comprise the modified ABCB9 gene, with a second parent plant, to obtain an F1 population,

b) optionally performing one or more rounds of selfing and/or crossing with a plant from said F1 population to obtain a further generation population,

c) selecting a plant that may comprise the modified ABCB9 gene.

The method for producing a plant that may comprise the modified ABCB9 gene can further also comprise the steps of:

d) testing the selected plant for showing CMV resistance;

e) selecting the plant if it is CMV resistant.

The second parent plant, as described in step a) of the method for producing a plant that may comprise the modified ABCB9 gene, as described herein, can also comprise the modified ABCB9 gene.

The invention also relates to a method for producing a plant that may comprise the modified EF1-α gene of the invention, said method which may comprise:

a) crossing a first parent plant which may comprise the modified EF1-α gene of the invention, with a second parent plant, to obtain an F1 population,

b) optionally performing one or more rounds of selfing and/or crossing with a plant from said F1 population to obtain a further generation population,

c) selecting a plant that may comprise the modified EF1-α gene.

The method for producing a plant that may comprise the modified EF1-α gene of the invention, can further also comprise the steps of:

d) testing the selected plant for showing CMV resistance;

e) selecting the plant if it is CMV resistant.

The second parent plant, as described in step a) of the method for producing a plant that may comprise the modified EF1-α gene, as described herein, can also comprise the modified EF1-α gene.

Also covered by the invention is a method for producing a plant that may comprise the modified ABCB9 gene of the invention, as described herein, wherein the first and/or the second parent plant also may comprise a modified EF1-α gene of the invention.

The invention also relates to a method for selecting a plant that may comprise the modified ABCB9 gene of the invention, said method which may comprise;

a) assaying nucleic acids of a plant for the presence of the modified ABCB9 gene of the invention,

b) identifying a plant that may comprise the modified ABCB9 gene, and selecting said plant.

The method for selecting a plant that may comprise the modified ABCB9 gene of the invention, can further also comprise;

c) testing the selected plant for CMV resistance,

d) selecting the plant if it shows CMV resistance.

As used herein, assaying nucleic acids of a plant may comprise isolating acids from a plant and analysing the isolated sample with a chosen method to detect the modified gene of interest. This method can be chosen from a group of methods that is well known in the art such as PCR, RT-PCR, antibody-assays, sequencing assays, genotyping assays, or any combination of these methods.

The invention also relates to a method for selecting a plant that may comprise the modified EF1-α gene of the invention, said method which may comprise;

a) assaying nucleic acids of a plant for the presence of the modified EF1-α gene of the invention,

b) identifying a plant that may comprise the modified EF1-α gene, and selecting said plant.

The method for selecting a plant that may comprise the modified EF1-α gene of the invention, can further also comprise;

c) testing the selected plant for CMV resistance,

d) selecting the plant if it shows CMV resistance.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### Examples

### Example 1: Bio assay for CMV resistance in Cucumber

Several internal cucumber plant lines with different combinations of the modified ABCB9 gene and the modified EF1-α gene were tested in a bio assay for showing resistance to CMV.

Cucumber seeds were sown and after germination the seedlings were grown for 6 days in a greenhouse compartment at a continuous temperature of 24° C. On day 7 after sowing, 24 plants per line were inoculated. Just before the inoculation, the plants were dusted with carborundum powder. Preparing the inoculum, fresh leaves infected with CMV, were ground and suspended in a phosphate buffer using a ratio of 3 ml of buffer for each gram of plant material. During the preparation of the inoculum, the buffer and the inoculum were being cooled. The inoculation itself was done by rubbing the leaves with a sponge that was dipped in the suspension made with the infected plant material. Directly after the inoculation, the plants were rinsed with plenty of water. The test CMV isolate that was used is known as CU-CMV-UK6 and is available from PRI-WUR in Wageningen. On day 9 after sowing, the inoculation was repeated. After the inoculation, plants were grown in a temperature regime of 18° C./20° C., night/day. Half of the plants for each line were assessed for symptoms of CMV infection on day 21 after sowing, and the other half of the plants were assessed on day 28 after sowing. The plants were scored for showing CMV infection symptoms according to classes 1 to 6, as presented in Table 2. For each line, 24 individual plants were scored by assessing the second leaf for symptoms. All scores per line were collected and averaged.

TABLE 2
Scores for the CMV resistance classes and relevant controls.
A lower CMV resistance score means a stronger resistance to CMV.

| | | Control |
|---|---|---|
| | | varieties |
| Class | Symptoms | Check |
| 1 | No visible mosaic pattern and growth inhibition | |
| 2 | Weak mosaic pattern and/or weak growth inhibition | |
| 3 | Mosaic pattern and/or growth inhibition visible | Gardon RZ |
| 4 | Clearly visible mosaic pattern and/or growth inhibition | |
| 5 | Strongly mosaic pattern, strong growth | |
| | inhibition and/or systemic symptoms | |
| 6 | Very strong mosaic pattern, strong growth | Ventura RZ |
| | inhibition, and/or systemic symptoms | |

The results of the bio assay on CMV resistance are presented in Table 3. The results are presented for different groups of cucumber plants.

TABLE 3
Average CMV scores and presence of the EF1-α gene
and ABCB9 gene SNPs. Note that a lower CMV resistance score
means a stronger resistance to CMV, see also the description
of the scores for CMV resistance classes in Table 2.

| | Average | | | |
|---|---|---|---|---|
| | CMV | | EF1-α | ABCB9 |
| | resistance | CMV | gene | gene |
| Cucumber Plants | score | phenotype | SNP | SNP |
| comprising wild type ABCB9 | 3.5 | Susceptible | C | G |
| and EF1-α genes | | | | |
| comprising only modified | 2.3 | Resistant | G | G |
| EF1-α gene homozygously | | | | |
| comprising only modified | 1.9 | Resistant | C | A |
| ABCB9 gene homozygously | | | | |
| comprising both modified | 1.1 | Resistant | G | A |
| genes ABCB9 and EF1-α | | | | |
| homozygously | | | | |

Example 2: Identification of the ABCB9 gene modification in Cucumis sativus

In a study to investigate the genetic cause of CMV resistance, an internal cucumber crossing population was developed. An analysis performed on this crossing population revealed the presence of a QTL on chromosome 6 causative of the CMV resistance. Fine-mapping using a F3 population and additional markers narrowed the QTL down to a smaller region.

In this smaller region of approximately 71 Kb, 12 mutations were found which are polymorphic between the 2 parental lines. Of these 12 mutations only 2 were found to be located within a gene and affect the gene function. One of the mutations was not predictive for the presence of CMV resistance. The other mutation was located in the ABCB9 gene and results in an amino acid change of the encoded protein. The mutation is a SNP and was identified at position 1282 in the wild type ABCB9 gene nucleotide sequence SEQ ID NO: 1 and constitutes a guanine (G) in the CMV susceptible wild type of the ABCB9 gene and a adenine (A) in the CMV resistant modified ABCB9 gene. This mutation leads to an substitution of the amino acid Valine (V), to the amino acid Methionine (M), on position 428 of the amino acid sequence of SEQ ID NO: 2. The position of the SNP lies in the so called Md1B domain. For various lines with different CMV resistance scores, genomic sequences of the ABCB9 gene were available in house and these were compared. The comparison of the sequences showed that in all the tested cucumber lines that showed resistance to CMV, the specific mutation in the ABCB9 was present.

FIG. 1 shows the coding sequence of ABCB9 gene and the accompanying protein sequence, both the CMV susceptible wild type and the CMV resistant modified version.

### Example 3: Identification of the EF1-α gene modification in Cucumis sativus

During the same study as described in Example 1, another QTL on chromosome 2 was found that confers resistance to CMV. An analysis performed on the crossing population revealed the presence of a QTL on chromosome 2 causative of the CMV resistance. Fine-mapping using a F3 population and additional markers narrowed the QTL down to a smaller region. In the smaller region of approximately 41Kb, 5 mutations were found, of which one was located in a gene and correlated with CMV resistance.

This mutation in the Elongation factor 1-alpha (EFI-a) gene gives an amino acid change. The mutation was identified as a SNP at position 1115 in the wild type EF1-α gene nucleotide sequence SEQ ID NO: 5 and constitutes a cytosine (C) in the CMV susceptible wild type of the EF1-α gene and a guanine (G) in the CMV resistant modified EF1-α gene. The mutation leads to an substitution of amino acid alanine (A), to amino acid glycine (G), on position 372 of SEQ ED NO: 5. The position of the SNP of the invention lies in the translation elongation factor 1 (TEF-1) protein domain family. For various lines with different CMV resistance scores, genomic sequences were available in house and these were compared. The comparison of the sequences showed that in all the tested cucumber lines that showed resistance to CMV, the specific mutation in the EF1-a was present. FIG. 2 shows the coding sequence of the EF1-α gene and the accompanying protein sequence, both the wild type and the modified version.

The invention is further described by the following numbered paragraphs:

1. A modified ABCB9 gene encoding a protein conferring resistance to CMV in a plant of the Cucurbitaceae, in particular a cucumber plant, in which the protein is expressed, characterized in that the gene comprises a nucleotide sequence selected from

a) a nucleotide sequence which encodes a protein comprising SEQ ID NO: 4;

b) a nucleotide sequence comprising SEQ ID NO: 3;

c) a nucleotide sequence encoding a protein derived by substitution, deletion and/or addition of one or more amino acids of the protein comprising SEQ ID NO: 4;

d) a nucleotide sequence that encodes a protein comprising an amino acid sequence, which is at least 85% identical to SEQ ID NO: 4;

e) a nucleotide sequence which is at least 85% identical to SEQ ID NO: 3;

f) a nucleotide sequence according to c) or d) wherein the protein comprises a methionine (M) on position 428 of SEQ ID NO: 4, or on a position corresponding thereto.

2. A protein, conferring resistance to CMV in a plant of the Cucurbitaceae, in particular a cucumber plant, in which the protein is expressed and which protein is encoded by the gene of paragraph 1.

3. A modified EF1-α gene encoding a protein conferring resistance to CMV in a cucumber plant, in which the protein is expressed, characterized in that the gene comprises a nucleotide sequence selected from

a) a nucleotide sequence which encodes a protein comprising SEQ ID NO: 8;

b) a nucleotide sequence comprising SEQ ID NO: 7;

c) a nucleotide sequence encoding a protein derived by substitution, deletion and/or addition of one or more amino acids of the protein comprising SEQ ID NO: 8,

d) a nucleotide sequence that encodes a protein comprising an amino acid sequence, which is at least 95% identical to SEQ ID NO: 8;

e) a nucleotide sequence which is at least 95% identical to SEQ ID NO: 7;

f) a nucleotide sequence according to c) or d) wherein the protein comprises a Glycine (G) on position 372 of SEQ ID NO: 8, or on a position corresponding thereto.

4. A protein, conferring resistance to CMV in a plant of the Cucurbitaceae, in particular a cucumber plant, in which the protein is expressed and which protein is encoded by the gene of paragraph 3.

5. A plant of the Cucurbitaceae, in particular a cucumber plant, comprising the modified ABCB9 gene of paragraph 1.

6. The plant of paragraph 5 comprising the modified ABCB9 gene homozygously, wherein the plant is resistant to CMV.

7. The plant of paragraph 5 and 6, further comprising the modified EF1-α gene of paragraph 3.

8. The plant of paragraph 5, 6 or 7, comprising the modified EF1-α gene, homozygously.

9. Part of a plant, of any of the paragraphs 5 to 8., which plant part comprises the modified ABCB9 gene of paragraph 1, and optionally the modified EF1-α gene of paragraph 3.

10. A seed comprising the modified ABCB9 gene of paragraph 1, and optionally the modified EF1-α gene of paragraph 3.

11. A tissue culture of a plant of any of the paragraphs 5 to 8, which tissue culture comprises the modified ABCB9 gene of paragraph 1, and optionally the modified EF1-α gene of paragraph 3.

12. A marker for identifying a cucumber plant comprising a modified ABCB9 gene, wherein the marker detects a modified nucleotide on position 1282 as compared to the wild type ABCB9 gene nucleotide sequence of SEQ ID NO: 1.

13. A marker of paragraph 12, wherein the marker detects an Adenine (A) on position 1282 of SEQ ID NO: 3.

14. A marker of paragraph 13, comprising SEQ ID NO: 11.

15. A marker for identifying a cucumber plant comprising a modified EF1-α gene, wherein the marker detects a modified nucleotide on position 1115 as compared to the wild type EF1-α gene nucleotide sequence of SEQ ID NO: 5.

16. A marker of paragraph 15, wherein the marker detects a Guanine (G) on position 1115 of SEQ ID NO: 7.

17. A marker of paragraph 16, comprising SEQ ID NO: 12.

18. Use of a marker of any of the paragraphs 12-17, for identifying a cucumber plant that comprises a modified ABCB9 gene and/or a modified EF1-α gene, and in particular for identifying a plant that is resistant to CMV.

19. A method for producing a plant that is CMV resistant by introducing the modified ABCB9 gene of paragraph 1 and optionally introducing the modified EF1-α gene of paragraph 3 into the genome of a plant.

20. A method for selecting a CMV resistant plant, comprising

a) identifying the presence of the modified ABCB9 gene of paragraph 1 and/or the presence of the modified EF-1 α gene of paragraph 3,

b) selecting a plant that comprises at least one of said modified genes;

c) optionally testing the selected plant for CMV resistance, and

d) selecting the plant if it shows CMV resistance as a CMV resistant plant.

21. Method of paragraph 20, wherein the identification is performed by using a marker as defined in paragraphs 12 to 14 and/or the marker as defined in paragraph 15 to 17.

22. A method for producing a plant that comprises the modified ABCB9 gene of paragraph 1, said method comprising:

a) crossing a first parent plant comprising the modified ABCB9 gene, of paragraph 1, with a second parent plant, to obtain an F1 population,

b) optionally performing one or more rounds of selfing and/or crossing with a plant from said F1 population to obtain a further generation population,

c) selecting a plant that comprises the modified ABCB9 gene.

23. A method for producing a plant that comprises the modified EF1-α gene of paragraph 3, said method comprising:

a) crossing a first parent plant comprising the modified EF1-α gene, of paragraph 3, with a second parent plant, to obtain an F1 population,

b) optionally performing one or more rounds of selfing and/or crossing with a plant from said F1 population to obtain a further generation population,

c) selecting a plant that comprises the modified EF1-α gene.

24. The method of paragraph 22 or 23, further comprising the steps of:

d) testing the selected plant for showing CMV resistance;

e) selecting the plant if it is CMV resistant.

25. Method of paragraph 22, wherein the second parent plant also comprises a modified ABCB9 gene of paragraph 1.

26. The method of paragraph 23, wherein the second parent plant also comprises the modified EF1-α gene of paragraph 3.

27. Method of paragraph 22, wherein the first and/or the second parent plant also comprises a modified EF1-α gene, of paragraph 3.

28. A method for selecting a plant that comprises the modified ABCB9 gene of paragraph 1 said method comprising;

a) assaying nucleic acids of a plant for the presence of the modified ABCB9 gene of paragraph 1;

b) identifying a plant that comprises the modified ABCB9 gene, and selecting said plant.

29. A method for selecting a plant that comprises the modified EF1-α gene, said method comprising;

a) assaying nucleic acids of a plant for the presence of the modified EF1-α gene of paragraph 3,

b) identifying a plant that comprises the modified EF1-α gene, and selecting said plant.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A modified ABCB9 gene encoding a protein conferring resistance to CMV in a plant of the Cucurbitaceae, in which the protein is expressed, characterized in that the gene comprises a nucleotide sequence of
a) a nucleotide sequence which encodes a protein comprising SEQ ID NO: 4;
b) a nucleotide sequence comprising SEQ ID NO: 3;
c) a nucleotide sequence encoding a protein derived by substitution, deletion and/or addition of one or more amino acids of the protein comprising SEQ ID NO: 4;
d) a nucleotide sequence that encodes a protein comprising an amino acid sequence, which is at least 85% identical to SEQ ID NO: 4;
e) a nucleotide sequence which is at least 85% identical to SEQ ID NO: 3; or
f) a nucleotide sequence according to c) or d) wherein the protein comprises a methionine (M) on position 428 of SEQ ID NO: 4, or on a position corresponding thereto.

2. A protein conferring resistance to CMV in a plant of the Cucurbitaceae, in which the protein is expressed and which protein is encoded by the gene of claim 1.

3. A modified EF1-α gene encoding a protein conferring resistance to CMV in a cucumber plant, in which the protein is expressed, characterized in that the gene comprises a nucleotide sequence of
a) a nucleotide sequence which encodes a protein comprising SEQ ID NO: 8;
b) a nucleotide sequence comprising SEQ ID NO: 7;
c) a nucleotide sequence encoding a protein derived by substitution, deletion and/or addition of one or more amino acids of the protein comprising SEQ ID NO: 8, d) a nucleotide sequence that encodes a protein comprising an amino acid sequence, which is at least 95% identical to SEQ ID NO: 8;
e) a nucleotide sequence which is at least 95% identical to SEQ ID NO: 7; or
f) a nucleotide sequence according to c) or d) wherein the protein comprises a Glycine (G) on position 372 of SEQ ID NO: 8, or on a position corresponding thereto.

4. A protein conferring resistance to CMV in a plant of the Cucurbitaceae, in which the protein is expressed and which protein is encoded by the gene of claim 3.

5. A plant of the Cucurbitaceae comprising the modified ABCB9 gene of claim 1.

6. The plant of claim 5 comprising the modified ABCB9 gene homozygously, wherein the plant is resistant to CMV.

7. The plant of claim 5, further comprising a modified EF1-α gene encoding a protein conferring resistance to CMV in a cucumber plant, in which the protein is expressed, characterized in that the gene comprises a nucleotide sequence of a) a nucleotide sequence which encodes a protein comprising SEQ ID NO: 8; b) a nucleotide sequence comprising SEQ ID NO: 7; c) a nucleotide sequence encoding a protein derived by substitution, deletion and/or addition of one or more amino acids of the protein comprising SEQ ID NO: 8, d) a nucleotide sequence that encodes a protein comprising an amino acid sequence, which is at least 95% identical to SEQ ID NO: 8; e) a nucleotide sequence which is at least 95% identical to SEQ ID NO: 7; or f) a nucleotide sequence according to c) or d) wherein the protein comprises a Glycine (G) on position 372 of SEQ ID NO:
8, or on a position corresponding thereto.

8. The plant of claim 5, comprising the modified EF1-α gene, homozygously.

9. A part of the plant of claim 5, which plant part comprises the modified ABCB9 gene and optionally a modified EF1-α gene encoding a protein conferring resistance to CMV in a cucumber plant, in which the protein is expressed, characterized in that the gene comprises a nucleotide sequence selected from a) a nucleotide sequence which encodes a protein comprising SEQ ID NO: 8; b) a nucleotide sequence comprising SEQ ID NO: 7; c) a nucleotide sequence encoding a protein derived by substitution, deletion and/or addition of one or more amino acids of the protein comprising SEQ ID NO: 8, d) a nucleotide sequence that encodes a protein comprising an amino acid sequence, which is at least 95% identical to SEQ ID NO: 8; e) a nucleotide sequence which is at least 95% identical to SEQ ID NO: 7; or f) a nucleotide sequence according to c) or d) wherein the protein comprises a Glycine (G) on position 372 of SEQ ID NO: 8, or on a position corresponding thereto.

10. A seed comprising the modified ABCB9 gene of claim 1, and optionally a modified EF1-α gene encoding a protein conferring resistance to CMV in a cucumber plant, in which the protein is expressed, characterized in that the gene comprises a nucleotide sequence of a) a nucleotide sequence which encodes a protein comprising SEQ ID NO: 8; b) a nucleotide sequence comprising SEQ ID NO: 7; c) a nucleotide sequence encoding a protein derived by substitution, deletion and/or addition of one or more amino acids of the protein comprising SEQ ID NO: 8, d) a nucleotide sequence that encodes a protein comprising an amino acid sequence, which is at least 95% identical to SEQ ID NO: 8; e) a nucleotide sequence which is at least 95% identical to SEQ ID NO: 7; or f) a nucleotide sequence according to c) or d) wherein the protein comprises a Glycine (G) on position 372 of SEQ ID NO: 8, or on a position corresponding thereto.

11. A tissue culture of the plant of claim 5, which tissue culture comprises the modified ABCB9 gene, and optionally a modified EF1-α gene encoding a protein conferring resistance to CMV in a cucumber plant, in which the protein is expressed, characterized in that the gene comprises a nucleotide sequence of a) a nucleotide sequence which encodes a protein comprising SEQ ID NO: 8; b) a nucleotide sequence comprising SEQ ID NO: 7; c) a nucleotide sequence encoding a protein derived by substitution, deletion and/or addition of one or more amino acids of the protein comprising SEQ ID NO: 8, d) a nucleotide sequence that encodes a protein comprising an amino acid sequence, which is at least 95% identical to SEQ ID NO: 8; e) a nucleotide sequence which is at least 95% identical to SEQ ID NO: 7; or f) a nucleotide sequence according to c) or d) wherein the protein comprises a Glycine (G) on position 372 of SEQ ID NO: 8, or on a position corresponding thereto.

12. A marker for identifying a cucumber plant comprising a modified ABCB9 gene, wherein the marker detects a modified nucleotide on position 1282 as compared to the wild type ABCB9 gene nucleotide sequence of SEQ ID NO: 1.

13. The marker of claim 12, wherein the marker detects an Adenine (A) on position 1282 of SEQ ID NO: 3.

14. The marker of claim 13, comprising SEQ ID NO: 11.

15. A marker for identifying a cucumber plant comprising a modified EF1-α gene, wherein the marker detects a modified nucleotide on position 1115 as compared to the wild type EF1-α gene nucleotide sequence of SEQ ID NO: 5.

16. The marker of claim 15, wherein the marker detects a Guanine (G) on position 1115 of SEQ ID NO: 7.

17. The marker of claim 16, comprising SEQ ID NO: 12.

18. A method of identifying a cucumber plant that comprises a modified ABCB9 gene and/or a modified EF1-α gene, and in particular for identifying a plant that is resistant to CMV, comprising detecting the plant with a marker that detects a modified nucleotide on position 1282 as compared to the wild type ABCB9 gene nucleotide sequence of SEQ ID NO: 1 or a marker that detects a modified nucleotide on position 1115 as compared to the wild type EF1-α gene nucleotide sequence of SEQ ID NO: 5.

19. A method for producing a plant that is CMV resistant by introducing the modified ABCB9 gene of claim 1 and optionally introducing a modified EF1-α gene encoding a protein conferring resistance to CMV in a cucumber plant, in which the protein is expressed, characterized in that the gene comprises a nucleotide sequence of a) a nucleotide sequence which encodes a protein comprising SEQ ID NO: 8; b) a nucleotide sequence comprising SEQ ID NO: 7; c) a nucleotide sequence encoding a protein derived by substitution, deletion and/or addition of one or more amino acids of the protein comprising SEQ ID NO: 8, d) a nucleotide sequence that encodes a protein comprising an amino acid sequence, which is at least 95% identical to SEQ ID NO: 8; e) a nucleotide sequence which is at least 95% identical to SEQ ID NO: 7; or f) a nucleotide sequence according to c) or d) wherein the protein comprises a Glycine (G) on position 372 of SEQ ID NO: 8, or on a position corresponding thereto into the genome of a plant.

20. A method for selecting a CMV resistant plant, comprising
a) identifying the presence of the modified ABCB9 gene of claim 1 and/or the presence of the modified EF-1 α gene of claim 3,
b) selecting a plant that comprises at least one of said modified genes;
c) optionally testing the selected plant for CMV resistance, and
d) selecting the plant if it shows CMV resistance as a CMV resistant plant.

21. The method of claim 20, wherein the identification is performed with a marker for identifying a cucumber plant comprising a modified ABCB9 gene, wherein the marker detects a modified nucleotide on position 1282 as compared to the wild type ABCB9 gene nucleotide sequence of SEQ ID NO: 1 or the marker detects a modified nucleotide on position 1115 as compared to the wild type EF1-α gene nucleotide sequence of SEQ ID NO: 5.

22. A method for producing a plant that comprises the modified ABCB9 gene of claim 1, said method comprising:
a) crossing a first parent plant comprising the modified ABCB9 gene, of claim 1, with a second parent plant, to obtain an F1 population,
b) optionally performing one or more rounds of selfing and/or crossing with a plant from said F1 population to obtain a further generation population,
c) selecting a plant that comprises the modified ABCB9 gene.

23. A method for producing a plant that comprises the modified EF1-α gene of claim 3, said method comprising:
a) crossing a first parent plant comprising the modified EF1-α gene, with a second parent plant, to obtain an F1 population,
b) optionally performing one or more rounds of selfing and/or crossing with a plant from said F1 population to obtain a further generation population,
c) selecting a plant that comprises the modified EF1-α gene.

24. The method of claim 22, further comprising the steps of:
d) testing the selected plant for showing CMV resistance;
e) selecting the plant if it is CMV resistant.

25. The method of claim 23, further comprising the steps of:
d) testing the selected plant for showing CMV resistance;
e) selecting the plant if it is CMV resistant.

26. The method of claim 22, wherein the second parent plant also comprises a modified ABCB9 gene of claim 1.

27. The method of claim 23, wherein the second parent plant also comprises the modified EF1-α gene of claim 3.

28. The method of claim 22, wherein the first and/or the second parent plant also comprises the modified EF1-α gene of claim 3.

29. A method for selecting a plant that comprises the modified ABCB9 gene of claim 1 said method comprising;
a) assaying nucleic acids of a plant for the presence of the modified ABCB9 gene of claim 1;
b) identifying a plant that comprises the modified ABCB9 gene, and selecting said plant.

30. A method for selecting a plant that comprises the modified EF1-α gene, said method comprising;
a) assaying nucleic acids of a plant for the presence of the modified EF1-α gene of claim 3,
b) identifying a plant that comprises the modified EF1-α gene, and selecting said plant.


SEQUENCE LISTING (12 sequences)

SEQ ID NO: 1 - DNA, 3813 bp, Cucumis sativus
  misc_feature 1..3813: note = Coding sequence ABCB9 wild type
  source 1..3813: mol_type = genomic DNA
atgggcggtggcggtggcggtggccacgaccacacaccttctccgccgaataatggacgaagcgatcagaaagttccattttataaactattcacattcgctgatcgctccgataatatattgatggccgtcggctccgtctgcgctgttgctaacggcctctctcagccaatcatgacgcttattttcggcaagatgatcgattcctttggctcctctaatcagtctaatgtcgttactcaagtttccaagatttccatagactttgtgtacctaggcattggtactggaattgcttcattcctacaggttgcttgttggatggtaacaggagaaagacaggcagcacgtattagagcattgtatttgaaaacaatactaagacaagacatcacatattttgacactgaaactacaactggagaagttattggtcgaatgtctggtgataccattcttatacaagatgccatgggagaaaaggtgggaaagttcatacagttgatgtcaactttctttggtggttttgtggttgccttcgcgaggggatggcttcttgctgtggttttactatcttgcattcctgcagttgttattgctggtggcaccacttcactcatcatgtctaaaatgtcaagccgtggacagattgcttatgcagaagcagggaatgtggttgaacaaaccgtaggagcgattagaacggtagcgtcattcacaggagagaaacaagcaatcgagaagtataacgagaagctgaaaatagcttataagtctactgttcaacaaggacttgccgcaggtttaggacttggcataatcttgttaattgcctttggaacttatgggcttgcagtatggtatggatctaagcttatcattcagaaggggtacaatggtggacaagttatcaatgttatatttgcaattatgactgggggaatgtcattgggccagacatcacctgtggtgaatgcatttgcttcagggcaggctgcagcttacaaaatgttcgagacgatcaaacgaaaaccaaaaattgattcctatgatgccagtggtatagcaccagaagacatacagggtgatattgaacttaaagatatctacttcagatatcctgctcggccagatgtgcagattttttcaggattctcgttgtttgttcccagtggaacaactgctgcgctcgttggacatagcggaagtggaaagtcgacagttatcagtttgctagaaagattctatgatcctgattctggtgaagtacttatagacggagtgaatttgaagcagtacaaacttagatggatcagagaaaaaatcggtcttgttagtcaagaacctatactctttacaactactataagggagaatatattgtatggaaaagacaatgcaacagaagaagaggtaagagcagcaattgagcttgcaaatgctgcaaagttcattgacaagctgcctaaggggcttgatacaatggtaggcgagcacgggactcaactatcaggtggacagaagcaaagaattgccatttcaagagccattttgaaaaatcccagaattctactccttgatgaagcaactagtgctttagattcagaatccgagcgtattgttcaagaagcactagtaagagtgatggcaaaccggacaaccgtggttgttgctcatcgcttgacaactataaggaattctgacaatatagcagtggtgcatcaagggaaactgctagagcaaggaacgcatgatgagctaatcaaaaatcctgatggagcttattctcaactagttcgtctgcaagaaggaacaacaacaggaacagaaacagaaactaatccgatcaacgatgccattgatctagacaaaacaatgggaagttctgccagcaagagaacatcagtgataagatctataagcaggacatcatcaggtagtcgacgttcattcacgatcaactttgccattccaggttcagttcatatccatgatcaagaaattgatgacgatggaccaaaaagaaatgacatggataagaaaaaacctaaacaggtttcaatgaaacgattggcaactttgaataagcctgagatgccagtgttgctccttggatgcattgcagcagttatgaatggcatggtctttcctatatttggcctcttactctcaagtgccattggaatgttttataagcctgcaagccagcttgagaaagaatccaaattttgggcgcttatttatctcggtctaggatgtctcacattctttgctttgcctacgcagaattactttttcgggattgcaggcggaaagttaatcgaaaggattcggtctttgacatttaaaaagattgtccaccaacagattagttattttgatgaccctgcaaacgcaagcggcgcaattggagcaaggttgtcaactgatgctgccacagtaagagggcttgtaggggatgcattggctttagtagtccaaaacattgcaaccatcacagctgggctaatcatagcattcacagctaactggatattagctttggtcattataggagtgtctcctttgctacttgtacaaggctacctccagaccaagttcacaaaagggttcagtgctgatgccaagatcatgtatgaagaagcgagtcaagtcgcgaacgacgcggtcggcagcatcagaacggttgcatcattttgctctgaaaagaaggtgatggatttatatgaaaagaaatgtgaggaccctgtcaaaaatggagttcgcctaggactcgtcagtggtgccggatttggcttttctttctttgctctcttctgcacaaatgccttttgcttctacattggatccattcttgtgaatcatggcaaggcaacatttcctgaagtcttcaaggtcttttttgctcttacaatttctgctatgggtgtttcccaaaccagtgccttggctcctgatagcagcaaagccaaggattccgcggcctctatatttgaaatactcgacagcaagcctaagattgattcaagtagtagtgaaggagtgacactcacctctgtaatcggcaatattgagtttgatcatgttagtttcaagtatccaacaaggccagatattcaaattttccgagatttatgcttaagaatcccgtctggaaagacagtggcattggttggagagagtggcagtggaaaatcaacagtaataagtctgatagaaagattttacgacccagactcgggacgaactcttctggacggagtagaaattcacaaattcaaactgagttggctaaggcaacaaatgggattagtgagtcaagaaccaatattattcaatgaaacaattcgatccaacatagcgtatggaaaaccagaaaatgcagcaagtgaagaagagataataggagcagcaaaagcagcaaatgcccacaatttcatatcttcattacccgaaggctacgaaacctcagtaggagaaagaggagttcaattatcaggaggccaaaaacagaggatagccattgctagagccattctaaagaacccaaaaattcttttgttggatgaagcaacgagcgcgttggacgccgagtcagagcgagttgtacaagatgcgttagatagagtgatggttaatagaacgacagtcgtcgttgcccatcggcttaccactattagaggggctgacattattgctgtggtgaaaaatggtgtgattgctgagaaaggaagccacgaggagctgatgaagatcagtgatggagcttatgcatcgttagtggcacttcattcaacttcttcaagtgtttag

SEQ ID NO: 2 - protein, 1270 aa, Cucumis sativus
  REGION 1..1270: note = Amino acid sequence ABCB9 protein wild type
MGGGGGGGHDHTPSPPNNGRSDQKVPFYKLFTFADRSDNILMAVGSVCAVANGLSQPIMTLIFGKMIDSFGSSNQSNVVTQVSKISIDFVYLGIGTGIASFLQVACWMVTGERQAARIRALYLKTILRQDITYFDTETTTGEVIGRMSGDTILIQDAMGEKVGKFIQLMSTFFGGFVVAFARGWLLAVVLLSCIPAVVIAGGTTSLIMSKMSSRGQIAYAEAGNVVEQTVGAIRTVASFTGEKQAIEKYNEKLKIAYKSTVQQGLAAGLGLGIILLIAFGTYGLAVWYGSKLIIQKGYNGGQVINVIFAIMTGGMSLGQTSPVVNAFASGQAAAYKMFETIKRKPKIDSYDASGIAPEDIQGDIELKDIYFRYPARPDVQIFSGFSLFVPSGTTAALVGHSGSGKSTVISLLERFYDPDSGEVLIDGVNLKQYKLRWIREKIGLVSQEPILFTTTIRENILYGKDNATEEEVRAAIELANAAKFIDKLPKGLDTMVGEHGTQLSGGQKQRIAISRAILKNPRILLLDEATSALDSESERIVQEALVRVMANRTTVVVAHRLTTIRNSDNIAVVHQGKLLEQGTHDELIKNPDGAYSQLVRLQEGTTTGTETETNPINDAIDLDKTMGSSASKRTSVIRSISRTSSGSRRSFTINFAIPGSVHIHDQEIDDDGPKRNDMDKKKPKQVSMKRLATLNKPEMPVLLLGCIAAVMNGMVFPIFGLLLSSAIGMFYKPASQLEKESKFWALIYLGLGCLTFFALPTQNYFFGIAGGKLIERIRSLTFKKIVHQQISYFDDPANASGAIGARLSTDAATVRGLVGDALALVVQNIATITAGLIIAFTANWILALVIIGVSPLLLVQGYLQTKFTKGFSADAKIMYEEASQVANDAVGSIRTVASFCSEKKVMDLYEKKCEDPVKNGVRLGLVSGAGFGFSFFALFCTNAFCFYIGSILVNHGKATFPEVFKVFFALTISAMGVSQTSALAPDSSKAKDSAASIFEILDSKPKIDSSSSEGVTLTSVIGNIEFDHVSFKYPTRPDIQIFRDLCLRIPSGKTVALVGESGSGKSTVISLIERFYDPDSGRTLLDGVEIHKFKLSWLRQQMGLVSQEPILFNETIRSNIAYGKPENAASEEEIIGAAKAANAHNFISSLPEGYETSVGERGVQLSGGQKQRIAIARAILKNPKILLLDEATSALDAESERVVQDALDRVMVNRTTVVVAHRLTTIRGADIIAVVKNGVIAEKGSHEELMKISDGAYASLVALHSTSSSV

SEQ ID NO: 3 - DNA, 3813 bp, Cucumis sativus
  misc_feature 1..3813: note = Coding sequence ABCB9 modified
  source 1..3813: mol_type = genomic DNA
atgggcggtggcggtggcggtggccacgaccacacaccttctccgccgaataatggacgaagcgatcagaaagttccattttataaactattcacattcgctgatcgctccgataatatattgatggccgtcggctccgtctgcgctgttgctaacggcctctctcagccaatcatgacgcttattttcggcaagatgatcgattcctttggctcctctaatcagtctaatgtcgttactcaagtttccaagatttccatagactttgtgtacctaggcattggtactggaattgcttcattcctacaggttgcttgttggatggtaacaggagaaagacaggcagcacgtattagagcattgtatttgaaaacaatactaagacaagacatcacatattttgacactgaaactacaactggagaagttattggtcgaatgtctggtgataccattcttatacaagatgccatgggagaaaaggtgggaaagttcatacagttgatgtcaactttctttggtggttttgtggttgccttcgcgaggggatggcttcttgctgtggttttactatcttgcattcctgcagttgttattgctggtggcaccacttcactcatcatgtctaaaatgtcaagccgtggacagattgcttatgcagaagcagggaatgtggttgaacaaaccgtaggagcgattagaacggtagcgtcattcacaggagagaaacaagcaatcgagaagtataacgagaagctgaaaatagcttataagtctactgttcaacaaggacttgccgcaggtttaggacttggcataatcttgttaattgcctttggaacttatgggcttgcagtatggtatggatctaagcttatcattcagaaggggtacaatggtggacaagttatcaatgttatatttgcaattatgactgggggaatgtcattgggccagacatcacctgtggtgaatgcatttgcttcagggcaggctgcagcttacaaaatgttcgagacgatcaaacgaaaaccaaaaattgattcctatgatgccagtggtatagcaccagaagacatacagggtgatattgaacttaaagatatctacttcagatatcctgctcggccagatgtgcagattttttcaggattctcgttgtttgttcccagtggaacaactgctgcgctcgttggacatagcggaagtggaaagtcgacagttatcagtttgctagaaagattctatgatcctgattctggtgaagtacttatagacggaatgaatttgaagcagtacaaacttagatggatcagagaaaaaatcggtcttgttagtcaagaacctatactctttacaactactataagggagaatatattgtatggaaaagacaatgcaacagaagaagaggtaagagcagcaattgagcttgcaaatgctgcaaagttcattgacaagctgcctaaggggcttgatacaatggtaggcgagcacgggactcaactatcaggtggacagaagcaaagaattgccatttcaagagccattttgaaaaatcccagaattctactccttgatgaagcaactagtgctttagattcagaatccgagcgtattgttcaagaagcactagtaagagtgatggcaaaccggacaaccgtggttgttgctcatcgcttgacaactataaggaattctgacaatatagcagtggtgcatcaagggaaactgctagagcaaggaacgcatgatgagctaatcaaaaatcctgatggagcttattctcaactagttcgtctgcaagaaggaacaacaacaggaacagaaacagaaactaatccgatcaacgatgccattgatctagacaaaacaatgggaagttctgccagcaagagaacatcagtgataagatctataagcaggacatcatcaggtagtcgacgttcattcacgatcaactttgccattccaggttcagttcatatccatgatcaagaaattgatgacgatggaccaaaaagaaatgacatggataagaaaaaacctaaacaggtttcaatgaaacgattggcaactttgaataagcctgagatgccagtgttgctccttggatgcattgcagcagttatgaatggcatggtctttcctatatttggcctcttactctcaagtgccattggaatgttttataagcctgcaagccagcttgagaaagaatccaaattttgggcgcttatttatctcggtctaggatgtctcacattctttgctttgcctacgcagaattactttttcgggattgcaggcggaaagttaatcgaaaggattcggtctttgacatttaaaaagattgtccaccaacagattagttattttgatgaccctgcaaacgcaagcggcgcaattggagcaaggttgtcaactgatgctgccacagtaagagggcttgtaggggatgcattggctttagtagtccaaaacattgcaaccatcacagctgggctaatcatagcattcacagctaactggatattagctttggtcattataggagtgtctcctttgctacttgtacaaggctacctccagaccaagttcacaaaagggttcagtgctgatgccaagatcatgtatgaagaagcgagtcaagtcgcgaacgacgcggtcggcagcatcagaacggttgcatcattttgctctgaaaagaaggtgatggatttatatgaaaagaaatgtgaggaccctgtcaaaaatggagttcgcctaggactcgtcagtggtgccggatttggcttttctttctttgctctcttctgcacaaatgccttttgcttctacattggatccattcttgtgaatcatggcaaggcaacatttcctgaagtcttcaaggtcttttttgctcttacaatttctgctatgggtgtttcccaaaccagtgccttggctcctgatagcagcaaagccaaggattccgcggcctctatatttgaaatactcgacagcaagcctaagattgattcaagtagtagtgaaggagtgacactcacctctgtaatcggcaatattgagtttgatcatgttagtttcaagtatccaacaaggccagatattcaaattttccgagatttatgcttaagaatcccgtctggaaagacagtggcattggttggagagagtggcagtggaaaatcaacagtaataagtctgatagaaagattttacgacccagactcgggacgaactcttctggacggagtagaaattcacaaattcaaactgagttggctaaggcaacaaatgggattagtgagtcaagaaccaatattattcaatgaaacaattcgatccaacatagcgtatggaaaaccagaaaatgcagcaagtgaagaagagataataggagcagcaaaagcagcaaatgcccacaatttcatatcttcattacccgaaggctacgaaacctcagtaggagaaagaggagttcaattatcaggaggccaaaaacagaggatagccattgctagagccattctaaagaacccaaaaattcttttgttggatgaagcaacgagcgcgttggacgccgagtcagagcgagttgtacaagatgcgttagatagagtgatggttaatagaacgacagtcgtcgttgcccatcggcttaccactattagaggggctgacattattgctgtggtgaaaaatggtgtgattgctgagaaaggaagccacgaggagctgatgaagatcagtgatggagcttatgcatcgttagtggcacttcattcaacttcttcaagtgtttag

SEQ ID NO: 4 - protein, 1270 aa, Cucumis sativus
  REGION 1..1270: note = Amino acid sequence ABCB9 protein modified
MGGGGGGGHDHTPSPPNNGRSDQKVPFYKLFTFADRSDNILMAVGSVCAVANGLSQPIMTLIFGKMIDSFGSSNQSNVVTQVSKISIDFVYLGIGTGIASFLQVACWMVTGERQAARIRALYLKTILRQDITYFDTETTTGEVIGRMSGDTILIQDAMGEKVGKFIQLMSTFFGGFVVAFARGWLLAVVLLSCIPAVVIAGGTTSLIMSKMSSRGQIAYAEAGNVVEQTVGAIRTVASFTGEKQAIEKYNEKLKIAYKSTVQQGLAAGLGLGIILLIAFGTYGLAVWYGSKLIIQKGYNGGQVINVIFAIMTGGMSLGQTSPVVNAFASGQAAAYKMFETIKRKPKIDSYDASGIAPEDIQGDIELKDIYFRYPARPDVQIFSGFSLFVPSGTTAALVGHSGSGKSTVISLLERFYDPDSGEVLIDGMNLKQYKLRWIREKIGLVSQEPILFTTTIRENILYGKDNATEEEVRAAIELANAAKFIDKLPKGLDTMVGEHGTQLSGGQKQRIAISRAILKNPRILLLDEATSALDSESERIVQEALVRVMANRTTVVVAHRLTTIRNSDNIAVVHQGKLLEQGTHDELIKNPDGAYSQLVRLQEGTTTGTETETNPINDAIDLDKTMGSSASKRTSVIRSISRTSSGSRRSFTINFAIPGSVHIHDQEIDDDGPKRNDMDKKKPKQVSMKRLATLNKPEMPVLLLGCIAAVMNGMVFPIFGLLLSSAIGMFYKPASQLEKESKFWALIYLGLGCLTFFALPTQNYFFGIAGGKLIERIRSLTFKKIVHQQISYFDDPANASGAIGARLSTDAATVRGLVGDALALVVQNIATITAGLIIAFTANWILALVIIGVSPLLLVQGYLQTKFTKGFSADAKIMYEEASQVANDAVGSIRTVASFCSEKKVMDLYEKKCEDPVKNGVRLGLVSGAGFGFSFFALFCTNAFCFYIGSILVNHGKATFPEVFKVFFALTISAMGVSQTSALAPDSSKAKDSAASIFEILDSKPKIDSSSSEGVTLTSVIGNIEFDHVSFKYPTRPDIQIFRDLCLRIPSGKTVALVGESGSGKSTVISLIERFYDPDSGRTLLDGVEIHKFKLSWLRQQMGLVSQEPILFNETIRSNIAYGKPENAASEEEIIGAAKAANAHNFISSLPEGYETSVGERGVQLSGGQKQRIAIARAILKNPKILLLDEATSALDAESERVVQDALDRVMVNRTTVVVAHRLTTIRGADIIAVVKNGVIAEKGSHEELMKISDGAYASLVALHSTSSSV

SEQ ID NO: 5 - DNA, 1344 bp, Cucumis sativus
  misc_feature 1..1344: note = Coding sequence EF1- gene wild type
  source 1..1344: mol_type = genomic DNA
atgggtaaagaaaagactcatatcaacatcgtggttattggccatgtcgactctggaaagtcgaccaccactggtcatcttatctacaagcttggagggattgacaagcgtgtgattgagagattcgagaaggaagccgctgagatgaacaagaggtcattcaagtatgcttgggtgctcgacaaacttaaggcagagcgtgaacgtggtattaccattgacattgccctgtggaagtttgagaccaccaagtactactgcacagtcattgatgctcccggacatcgtgactttatcaagaacatgattactggaacctcacaggctgactgtgctgtcctcattattgactccaccactggtggttttgaggctggtatttccaaggatggtcagacccgtgagcacgctcttcttgctttcacccttggtgtcaagcaaatgatctgctgctgcaacaagatggatgccaccacacccaaatactccaaggcaaggtacgatgaaattgtgaaggaagtctcatcatacctcaagaaggtcggatacaacccagacaaaatccccttcgttcccatctctggtttcgagggtgacaacatgattgagaggtccaccaaccttgattggtacaagggaccaacccttcttgaggctcttgacttgatctctgagcccaagaggccctcagacaagcccctccgtctcccacttcaggacgtttacaagattggtggtattggaactgtccctgttggacgtgttgaaactggtgtcctcaagcctggtatggttgttaccttcggaccaactggactgaccactgaagttaagtctgttgaaatgcaccacgagtctctcccagaagctttgccaggtgacaatgttggcttcaacgtcaagaacgttgctgtcaaggatctcaagcgtggtttcgttgcctcaaactccaaggatgaccctgccaaggaagctgctaactttacatctcaggttatcatcatgaaccaccctggtcagattggtaatggttatgctccagtccttgattgccacacttcccacattgctgttaagttcgctgagatcctcaccaaaatcgatcgtcgatctgctaaggagcttgagaaggagcccaagttcttgaagaatggagatgccggtatggttaagatgattccaaccaaacccatggttgtggagaccttctctacatacccaccattgggtcgatttgctgtccgtgatatgcgtcaaaccgttgctgttggtgttatcaagagtgtggagaagaaggacccaacaggagccaaggtcaccaagtccgctgtgaagaagaaatag

SEQ ID NO: 6 - protein, 447 aa, Cucumis sativus
  REGION 1..447: note = Amino acid sequence EF1- protein wild type
MGKEKTHINIVVIGHVDSGKSTTTGHLIYKLGGIDKRVIERFEKEAAEMNKRSFKYAWVLDKLKAERERGITIDIALWKFETTKYYCTVIDAPGHRDFIKNMITGTSQADCAVLIIDSTTGGFEAGISKDGQTREHALLAFTLGVKQMICCCNKMDATTPKYSKARYDEIVKEVSSYLKKVGYNPDKIPFVPISGFEGDNMIERSTNLDWYKGPTLLEALDLISEPKRPSDKPLRLPLQDVYKIGGIGTVPVGRVETGVLKPGMVVTFGPTGLTTEVKSVEMHHESLPEALPGDNVGFNVKNVAVKDLKRGFVASNSKDDPAKEAANFTSQVIIMNHPGQIGNGYAPVLDCHTSHIAVKFAEILTKIDRRSAKELEKEPKFLKNGDAGMVKMIPTKPMVVETFSTYPPLGRFAVRDMRQTVAVGVIKSVEKKDPTGAKVTKSAVKKK

SEQ ID NO: 7 - DNA, 1344 bp, Cucumis sativus
  misc_feature 1..1344: note = Coding sequence EF1- gene modified
  source 1..1344: mol_type = genomic DNA
atgggtaaagaaaagactcatatcaacatcgtggttattggccatgtcgactctggaaagtcgaccaccactggtcatcttatctacaagcttggagggattgacaagcgtgtgattgagagattcgagaaggaagccgctgagatgaacaagaggtcattcaagtatgcttgggtgctcgacaaacttaaggcagagcgtgaacgtggtattaccattgacattgccctgtggaagtttgagaccaccaagtactactgcacagtcattgatgctcccggacatcgtgactttatcaagaacatgattactggaacctcacaggctgactgtgctgtcctcattattgactccaccactggtggttttgaggctggtatttccaaggatggtcagacccgtgagcacgctcttcttgctttcacccttggtgtcaagcaaatgatctgctgctgcaacaagatggatgccaccacacccaaatactccaaggcaaggtacgatgaaattgtgaaggaagtctcatcatacctcaagaaggtcggatacaacccagacaaaatccccttcgttcccatctctggtttcgagggtgacaacatgattgagaggtccaccaaccttgattggtacaagggaccaacccttcttgaggctcttgacttgatctctgagcccaagaggccctcagacaagcccctccgtctcccacttcaggacgtttacaagattggtggtattggaactgtccctgttggacgtgttgaaactggtgtcctcaagcctggtatggttgttaccttcggaccaactggactgaccactgaagttaagtctgttgaaatgcaccacgagtctctcccagaagctttgccaggtgacaatgttggcttcaacgtcaagaacgttgctgtcaaggatctcaagcgtggtttcgttgcctcaaactccaaggatgaccctgccaaggaagctgctaactttacatctcaggttatcatcatgaaccaccctggtcagattggtaatggttatgctccagtccttgattgccacacttcccacattgctgttaagttcgctgagatcctcaccaaaatcgatcgtcgatctggtaaggagcttgagaaggagcccaagttcttgaagaatggagatgccggtatggttaagatgattccaaccaaacccatggttgtggagaccttctctacatacccaccattgggtcgatttgctgtccgtgatatgcgtcaaaccgttgctgttggtgttatcaagagtgtggagaagaaggacccaacaggagccaaggtcaccaagtccgctgtgaagaagaaatag

SEQ ID NO: 8 - protein, 447 aa, Cucumis sativus
  REGION 1..447: note = Amino acid sequence EF1- protein modified
MGKEKTHINIVVIGHVDSGKSTTTGHLIYKLGGIDKRVIERFEKEAAEMNKRSFKYAWVLDKLKAERERGITIDIALWKFETTKYYCTVIDAPGHRDFIKNMITGTSQADCAVLIIDSTTGGFEAGISKDGQTREHALLAFTLGVKQMICCCNKMDATTPKYSKARYDEIVKEVSSYLKKVGYNPDKIPFVPISGFEGDNMIERSTNLDWYKGPTLLEALDLISEPKRPSDKPLRLPLQDVYKIGGIGTVPVGRVETGVLKPGMVVTFGPTGLTTEVKSVEMHHESLPEALPGDNVGFNVKNVAVKDLKRGFVASNSKDDPAKEAANFTSQVIIMNHPGQIGNGYAPVLDCHTSHIAVKFAEILTKIDRRSGKELEKEPKFLKNGDAGMVKMIPTKPMVVETFSTYPPLGRFAVRDMRQTVAVGVIKSVEKKDPTGAKVTKSAVKKK

SEQ ID NO: 9 - DNA, 201 bp, Cucumis sativus
  misc_feature 1..201: note = marker for the wild type ABCB9 gene
  source 1..201: mol_type = genomic DNA
tgctgcgctcgttggacatagcggaagtggaaagtcgacagttatcagtttgctagaaagattctatgatcctgattctggtgaagtacttatagacggagtgaatttgaagcagtacaaacttagatggatcagagaaaaaatcggtcttgttagtcaagaacctatactctttacaactactataagggagaatatatt

SEQ ID NO: 10 - DNA, 201 bp, Cucumis sativus
  misc_feature 1..201: note = marker for the wild type EF1- gene
  source 1..201: mol_type = genomic DNA
tgtagagaaggtctccacaaccatgggtttggttggaatcatcttaaccataccggcatctccattcttcaagaacttgggctccttctcaagctccttaccagatcgacgatcrattttggtgaggatctcagcgaacttaacagcaatgtgggaagtgtggcaatcaaggactggagcataaccattaccaatctgacc

SEQ ID NO: 11 - DNA, 201 bp, Cucumis sativus
  misc_feature 1..201: note = marker for the modified ABCB9 gene
  source 1..201: mol_type = genomic DNA
tgctgcgctcgttggacatagcggaagtggaaagtcgacagttatcagtttgctagaaagattctatgatcctgattctggtgaagtacttatagacggaatgaatttgaagcagtacaaacttagatggatcagagaaaaaatcggtcttgttagtcaagaacctatactctttacaactactataagggagaatatatt

SEQ ID NO: 12 - DNA, 201 bp, Cucumis sativus
  misc_feature 1..201: note = marker for the modified EF1- gene
  source 1..201: mol_type = genomic DNA
tgtagagaaggtctccacaaccatgggtttggttggaatcatcttaaccataccggcatctccattcttcaagaacttgggctccttctcaagctccttagcagatcgacgatcrattttggtgaggatctcagcgaacttaacagcaatgtgggaagtgtggcaatcaaggactggagcataaccattaccaatctgacc